(19) United States
(12) Patent Application Publication
(10) Pub. No.: US 2022/0305020 A1
(43) Pub. Date: Sep. 29, 2022

(54) THE USE OF PARAXANTHINE TO REDUCE EXERCISE-INDUCED MENTAL FATIGUE

(71) Applicant: Ingenious Ingredients, LP, Lewisville, TX (US)

(72) Inventors: Ralf Jäger, Whitefish Bay, WI (US); Martin Purpura, Spring, TX (US); Shawn Wells, Frisco, TX (US); Kylin Liao, Plano, TX (US)

(21) Appl. No.: 17/706,183

(22) Filed: Mar. 28, 2022

Related U.S. Application Data
(60) Provisional application No. 63/166,494, filed on Mar. 26, 2021.

(51) Int. Cl. A61K 31/522; A61K 45/06; A23L 33/10; A61P 25/26

(52) U.S. Cl. CPC A61K 31/522; A61K 45/06; A23L 33/10; A61P 25/26

(57) ABSTRACT

Disclosed herein are compositions and methods for attenuating stress-induced mental fatigue in subject in need thereof by administering to the subject a composition comprising from about 2 mg to about 800 mg of paraxanthine. In certain embodiments, paraxanthine is present in the composition in amount from about 20 mg to about 600 mg. In further embodiments, paraxanthine is present in the composition in amount from about 50 mg to about 400 mg. According to certain embodiments, fatigue attenuated by the instantly disclosed method is a result of physical stress on the subject. Physical stresses that may result in mental fatigue include, but are not limited to, periods of intense exercise.

### CROSS-REFERENCE TO RELATED APPLICATION(S)

This application claims priority to U.S. Provisional Application No. 63/166,494 filed Mar. 26, 2021 and entitled “THE USE OF PARAXANTHINE TO REDUCE EXERCISE-INDUCED MENTAL FATIGUE,” which is hereby incorporated by reference in its entirety under 35 U.S.C. § 119(e).

### TECHNICAL FIELD

The disclosed technology relates generally to compositions, methods, and system for utilizing paraxanthine alone and in combination for use attenuating stress-induced mental fatigue. More particularly, the disclosure relates to paraxanthine and other compounds, whether produced synthetically or derived from natural sources, and use of these chemical compounds to provide physiological benefits, which may vary according to paraxanthine concentration and the presence of synergists and antagonists.

### BACKGROUND

Caffeine is found in many beverages, including coffee, tea, energy drinks and colas, and in products containing cocoa or chocolate. It is also found in medications and dietary supplements, including supplements aimed at reducing fatigue. However, undesirable side effects, physical tolerance, and at high doses toxicity limit the usefulness of caffeine in countering the effects of prolonger physical exertion or psychological stress on mental fatigue.

Thus, there is a need in the art to identify alternative chemical compounds and mixtures thereof that may provide benefits in attenuating fatigue. It is also desirable to provide chemical compounds and mixtures thereof that may be used to provide a variety of benefits, varying by concentration, thus requiring production of fewer materials.

### BRIEF SUMMARY

Disclosed herein are compositions and methods for attenuating stress-induced mental fatigue in subject in need thereof by administering to the subject a composition comprising from about 2 mg to about 800 mg of paraxanthine. In certain embodiments, paraxanthine is present in the composition in amount from about 20 mg to about 600 mg. In further embodiments, paraxanthine is present in the composition in amount from about 50 mg to about 400 mg.

According to certain embodiments, fatigue attenuated by the instantly disclosed method is a result of physical stress on the subject. Physical stresses that may result in mental fatigue include, but are not limited to, periods of intense exercise.

According to further embodiments, fatigue attenuated by the instantly disclosed method is the result of a psychological stress. In certain implementations, the psychological stress is the result of a period of intense cognitively demanding task such as a timed examination. In further embodiments, the psychological stress is the result of periods of intense emotion (e.g. a trauma).

Further disclosed herein is a method of enhancing the stress resiliency in a subject in need thereof by administering to the subject a composition comprising from about 2 mg to about 800 mg of paraxanthine. In certain embodiments, administration of the composition to the subject increases resiliency exercise-induced stress. In further embodiments, the composition to the subject increases resiliency psychologically-induced stress. In certain embodiments, administration of the composition to the subject increases BDNF level in the subject and wherein BDNF levels are increased by from about 5% to about 40%.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF DRAWINGS

FIG. 1 shows a schematic representation of the experimental design, according to certain embodiments.

FIG. 2 shows data representing changes in BCST performance upon administration of the disclosed compositions, according to certain embodiments.

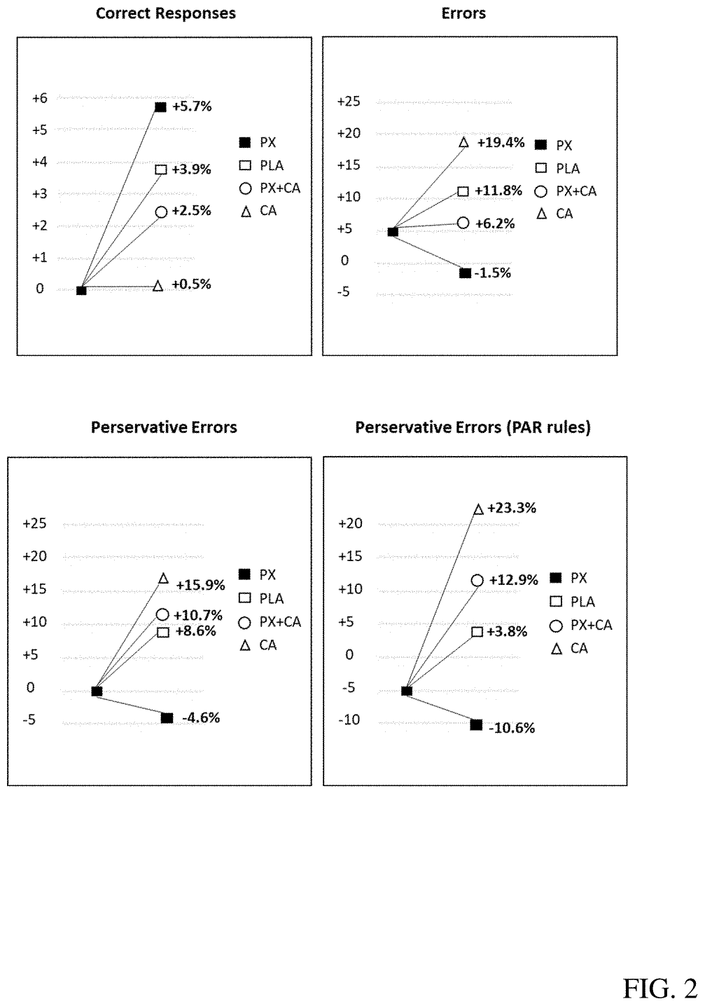

FIG. 3 shows data representing changes in PVTT performance upon administration of the disclosed compositions, according to certain embodiments.

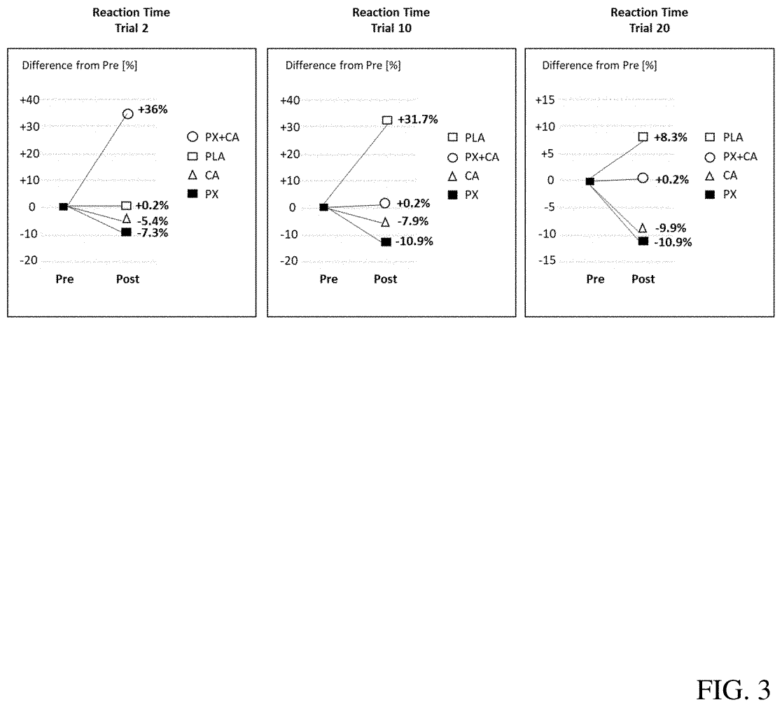

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause unacceptable adverse side effects. The specific therapeutically effective dose level for any particular patient will depend upon a variety of factors including the disorder being treated and the severity of the disorder; the specific composition employed; the age, body weight, general health, sex and diet of the subject; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “cognitive function” refers to any higher order intellectual brain process or brain state, respectively, involved in learning and/or memory including, but not limited to, attention, information acquisition, information processing, working memory, short-term memory, long-term memory, anterograde memory, retrograde memory, memory retrieval, discrimination learning, decision-making, inhibitory response control, attentional set-shifting, delayed reinforcement learning, reversal learning, the temporal integration of voluntary behavior, and expressing an interest in one's surroundings and self-care, speed of processing, reasoning and problem solving and social cognition.

As used herein, “attenuating stress-induced mental fatigue” means a measurable improvement of at least one deficit associated with mental fatigue. In certain embodiments, the one or more deficit is one or more cognitive functions. A person of skill in the art will select the known methods of measuring the improvement of cognitive functions.

As used herein, “mental fatigue” refers to the temporary decrease in cognitive function and/or physical performance that accompanies a period of intense exertion. In certain embodiments, the period of intense exertion involves performing a cognitively taxing task (e.g., taking a timed examination). In further embodiments, the period of intense exertion involves performing a physically taxing task.

“Stress related disorders” may refer collectively to maladies characterized by a state of hyper- or hypo-arousal with hyper- and hypo-vigilance. Stress related disorders include, without limitation: depression, major depressive disorder (MDD), anxiety disorder, panic disorder (episodic paroxysmal anxiety), panic attack, obsessive compulsive disorder, social anxiety disorder, phobic anxiety disorders (e.g., acrophobia, claustrophobia, agoraphobia, social phobia, and other phobias), posttraumatic stress disorder (PTSD), acute stress disorder, and obsessive compulsive disorder. Stress-related disorders may also include non-psychiatric disorders such as hypertension, inflammatory bowel syndrome and the like. Stress related disorders may be characterized low mental allostatic capacity and too great of allostatic load, a lack of mental resilience and low levels of BDNF, NGF and mTOR. Stress related disorders may also be characterized by aberrant noradrenergic, serotonergic, cholinergic, dopaminergic, and/or glutaminergic tone. In further embodiments, stress related disorders are characterized by energetic dysfunction of insufficient cellular energy states (ICE) with mitochondrial dysfunction, glucose intolerance as a result, which in turn gives rise to cholinergic depletion and dysregulation of other neurotransmitter systems as well as ATP.

As used herein, “stressor” refers to any stimulus that causes a stress reaction in a subject. The stressor may be an external stimulus. Examples of stressors include, without limitation, sensory inputs (e.g., pain, bright light, noise, and the like), trauma, conflict, social, interpersonal, cognitive, and the like. In certain embodiments, a stressor may be a period of intense mental exertion. In further embodiments, a stressor may be a period of intense physical exertion, such as that accompanying intense physical exercise.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is substantially free of particles would either completely lack particles, or so nearly completely lack particles that the effect would be the same as if it completely lacked particles. In other words, a composition that is substantially free of an ingredient or element may still actually contain such item as long as there is no measurable effect thereof.

This disclosure relates to the use chemical compositions comprising paraxanthine. In certain embodiments, paraxanthine is produced naturally. In further embodiments, paraxanthine is produced synthetically. In certain embodiments, the composition comprises other chemicals, including paraxanthine congeners or analogs, to provide a plurality of desirable effects. Paraxanthine analogs may include, but are not limited to, caffeine, methyl caffeine, theobromine, theophylline, liberine and methylliberine, and their variants. Other suitable actives may include one or more fatigue reducing compounds such, L-Theanine, blood flow enhancing ingredients including nitric stimulating nutrients such as L-Arginine, L-Citrulline, Ginkgo Biloba, neurotransmitter enhancing ingredients such as choline, phosphatidylserine, or ingredients known to protect the brain from oxidative stress and/or inflammation such as creatine, omega-3 fatty acids, beta-alanine; L-tyrosine; N-Acetyl-L-tyrosine; L-Ornithine; L-ornithine-L-aspartate; Melissa officinalis (Lemon Balm); pyrroloquinoline quinone; L-taurine; arginine alpha-ketoglutarate and blueberries.

According to certain further embodiments, the composition further comprises an anti-cortisol agent. In exemplary implementations, the anti-cortisol agent is one or more of: phosphatidylserine, rhodiola, ashwagandha, magnolia, holy basil, omega 3s, and/or l-theanine.

According to certain embodiments, the composition does not include caffeine. In certain implementations, the subject abstains from consuming caffeine during the steps of the disclosed method.

According to certain embodiments, fatigue attenuated by the instantly disclosed method is a result of physical stress on the subject. Physical stresses that may result in mental fatigue include, but are not limited to, periods of intense exercise.

According to further embodiments, fatigue attenuated by the instantly disclosed method is the result of a psychological stress. In certain implementations, the psychological stress is the result of a period of intense cognitively demanding task such as a timed examination.

In further embodiments, the psychological stress is the result of periods of intense emotion (e.g. a trauma). In further embodiments, the psychological stress may result from forms of neglect that may result in diminished/impaired cognitive activity. Psychological stress in these embodiments are characterized (often in depressed subjects) by low levels of BDNF and reduced neuroplasticity. Such subjects may experience destructive mental loops replaying the same things over and over which may be viewed as a form of self-abuse/self inflicted trauma.

In certain embodiments, the stress resulting in mental fatigue is an acute stress. These embodiments may be characterized by a highly intense stress over a finite period of time. In certain implementations, the acute stress lasts for 5-60 minutes. In further embodiments, the acute stress lasts for about 1 to about 8 hours.

According to certain further embodiments, the stress resulting in mental fatigue is a chronic stress. In these embodiments, stress may last for days, weeks, months or longer.

In certain embodiments, the composition is administered to the subject while the subject is experiencing a stressor. In certain further implementations, the composition is administered prior to the occurrence of stress in the subject (e.g., in anticipation of a coming stressor). In yet further implementations, the composition is administered following the conclusion of the stressor. In still further implementations, the composition is administered in some combination of the foregoing.

Further disclosed herein is a method of enhancing the stress resiliency in a subject in need thereof by administering to the subject a composition comprising from about 2 mg to about 800 mg of paraxanthine. In certain implementations, administration of the composition to the subject increases resiliency exercise-induced stress. In further implementations, administration of the composition to the subject increases resiliency psychologically-induced stress.

According to certain embodiments, administration of the composition to the subject increases brain derived neurotrophic factor (BDNF) levels in the subject. In exemplary implementations, BDNF levels are increased by from about 5% to about 40%. In further embodiments, BDNF levels are increased by at least about 15%. In further embodiments, administration of the composition to the subject increases other neurotrophic factors such as neuronal growth factor (NGF). In still further embodiments, administration of the composition to the subject increases levels of mTOR in the CNS.

According to certain further embodiments, administration of the composition to the subject increases levels of catalase and/or glutathione in the subject.

Further disclosed herein is a method of preventing or treating a stress related disorder in a subject in need thereof comprising administering to the subject a composition comprising from about 2 mg to about 800 mg of paraxanthine. In certain implementations, the stress related disorder is selected from depression, major depressive disorder (MDD), anxiety disorder, panic disorder (episodic paroxysmal anxiety), panic attack, obsessive compulsive disorder, social anxiety disorder, phobic anxiety disorders, posttraumatic stress disorder (PTSD), acute stress disorder, obsessive compulsive disorder, hypertension, and inflammatory bowel syndrome. In certain embodiments, the subject may suffer from, or be at risk of suffering from, a combination of the foregoing disorders.

Further disclosed herein is a method of attenuating fatigue associated oxidative stress in a subject in need thereof by administering to the subject a composition comprising from about 2 mg to about 800 mg of paraxanthine. In certain implementations of these embodiments, administration of the composition to the subject increases levels of catalase in the subject. In exemplary implementations, catalase levels are increased in the subject by from about 5% to about 70%. In further implementations, administration of the composition to the subject increases glutathione in the subject. In exemplary implementations, wherein glutathione levels are increased in the subject by from about 3% to about 30%.

Further disclosed herein is a method of attenuating the effects of chronic stress on a subject by administering to the subject a composition comprising from about 2 mg to about 800 mg of paraxanthine. In certain implementations, the composition further comprises an anti-cortisol agent. According to certain embodiments, the anti-cortisol agent is one or more of: phosphatidylserine, rhodiola, ashwagandha, magnolia, holy basil, Omega 3s, and/or l-theanine.

In certain embodiments, the disclosed compositions are nutraceutical compositions. Exemplary nutraceutical compositions of the present disclosure may be formulated or used as a standalone composition, or as a nutritional or bioactive component in food, a functional food, a beverage, a bar, a food flavor, a medical food, a dietary supplement, or an herbal product. A medium generally accepted in the art includes all pharmaceutically or nutraceutically acceptable carriers, diluents or excipients therefor.

Further disclosed herein is a method for attenuating stress-induced mental fatigue in a subject by providing the subject with a composition comprising about 2 mg to about 800 mg of paraxanthine. Although doses will vary from subject to subject, suitable daily doses are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses In certain aspects, paraxanthine is present in the composition in amount from about 20 mg to about 600 mg. In further aspects, paraxanthine is present in the composition in amount from about 50 mg to about 400 mg.

According to certain embodiments of the disclosed method, the composition is administered in a therapeutically effective amount. In further embodiments, the composition is administered in a prophylactically effective amount.

An advantage of using the invention may be the reduced likelihood that a person develops a tolerance to chemical compositions in accordance with the principles of the invention. That is, a person may not become desensitized to the effects induced.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect, but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Various aspects and embodiments of the present invention are defined by the following numbered clauses:

1. A method for attenuating stress-induced mental fatigue in subject in need thereof, comprising:
administering to the subject a composition comprising from about 2 mg to about 800 mg of paraxanthine.
2. The method of clause 1, wherein paraxanthine is present in the composition in amount from about 20 mg to about 600 mg.
3. The method of clause 2, wherein paraxanthine is present in the composition in amount from about 50 mg to about 400 mg.
4. The method of any of clauses 1-3, wherein the stress is physical stress.
5. The method of any of clauses 1-3, wherein the stress is psychological stress.
6. The method of clause 5, wherein the stress is an acute stress.
7. The method of clause 5, wherein the stress is a chronic stress.
8. The method of any of clauses 1-5, wherein the composition further comprises one or more additional active agents.
9. The method of clause 8, wherein in the one or more active agents are selected from L-Theanine, blood flow enhancing ingredients including nitric stimulating nutrients such as L-Arginine, L-Citrulline, Ginkgo Biloba, neurotransmitter enhancing ingredients such as choline, phosphatidylserine, or ingredients known to protect the brain from oxidative stress and/or inflammation such as creatine, omega-3 fatty acids, beta-alanine; L-tyrosine; N-Acetyl-L-tyrosine; L-Ornithine; L-ornithine-L-aspartate; Melissa officinalis (Lemon Balm); pyrroloquinoline quinone; L-taurine; arginine alpha-ketoglutarate, and blueberries.
10. The method of any preceding clause, wherein the composition is administered prior to the onset of stress in the subject.
11. The method of clause 10, wherein the composition further comprises an anti-cortisol agent.
12. The method of clause 11, wherein the anti-cortisol agent is one or more of: phosphatidylserine, rhodiola, ashwagandha, magnolia, holy basil, omega 3s, and/or l-theanine.
13. The method of any preceding clause, wherein the composition is administered during to the occurrence of stress in the subject.
14. The method of any preceding clause, wherein the composition does not include caffeine.
15. The method of clause 14, wherein the subject abstains from consuming caffeine.
16. The method of any preceding clause, wherein the composition is administer following the occurrence of stress in the subject.
17. The method of any preceding clause, wherein the paraxanthine is synthetic.
18. The method of any preceding clause, wherein the paraxanthine is derived from a natural source.
19. The method of any preceding clause wherein the composition is a dietary supplement.
20. The method of clause 19, wherein the dietary supplement is powder or a capsule.
21. The method of any preceding clause, wherein the composition is a functional food.
22. The method of clause 21, wherein the functional food is a beverage, nutrition bar, yoghurt, or cereal.
23. A nutritional supplement to prevent mental fatigue induced by a stressor comprising from about 2 mg to about 800 mg paraxanthine and a nutraceutically acceptable carrier thereof.
24. The nutritional supplement of clause 23 wherein the paraxanthine is present in amount from about 20 mg to about 600 mg.
25. The nutritional supplement of clause 23 wherein the paraxanthine is present in amount from about 50 mg to about 400 mg.
26. The nutritional supplement of any of clauses 23-25, wherein the nutritional supplement is a dietary supplement.
27. The nutritional supplement of clause 26, wherein the dietary supplement is powder or a capsule.
28. The nutritional supplement any of clauses 23-25, wherein the nutritional supplement is a functional food.
29. The nutritional supplement of clause 28, wherein the functional food is a beverage, nutrition bar, yoghurt, or cereal.
30. The nutritional supplement any of clauses 23-25, further comprises one or more compounds selected from the list consisting of: L-Theanine, blood flow enhancing ingredients including nitric stimulating nutrients such as L-Arginine, L-Citrulline, Ginkgo Biloba, neurotransmitter enhancing ingredients such as choline, phosphatidylserine, or ingredients known to protect the brain from oxidative stress and/or inflammation such as creatine, omega-3 fatty acids, beta-alanine; L-tyrosine; N-Acetyl-L-tyrosine; L-Ornithine; L-ornithine-L-aspartate; Melissa officinalis (Lemon Balm); pyrroloquinoline quinone; L-taurine; arginine alpha-ketoglutarate and blueberries.
31. The nutritional supplement of clause 30, further comprising an anti-cortisol agent.
32. The nutritional supplement of clause 31, wherein the anti-cortisol agent is one or more of: phosphatidylserine, rhodiola, ashwagandha, magnolia, holy basil, omega 3s, and/or l-theanine.
33. The nutritional supplement of any of clauses 23-30, wherein the paraxanthine is derived from a natural source.
34. The nutritional supplement of any of clauses 23-30, wherein the paraxanthine is synthetic.
35. The nutritional supplement of any of clauses 23-34, wherein the stressor is physical.
36. The nutritional supplement of any of clauses 23-34, wherein the stressor is mental.
37. A method of enhancing the stress resiliency in a subject in need thereof, comprising: administering to the subject a composition comprising from about 2 mg to about 800 mg of paraxanthine.
38. The method of clause 37, wherein administration of the composition to the subject increases resiliency exercise-induced stress.
39. The method of clause 38, wherein administration of the composition to the subject increases resiliency psychologically-induced stress.
40. The method of any of clauses 37-39, wherein administration of the composition to the subject increases BDNF level in the subject.
41. The method of clause 40, wherein BDNF levels are increased by from about 5% to about 40%.
42. The method of clause 41, wherein BDNF levels are increased by at least about 15%.
43. The method of any of clauses 37-40, wherein administration of the composition to the subject increases levels of catalase and/or glutathione in the subject.
44. A method of preventing or treating a stress related disorder in a subject in need thereof comprising administering to the subject a composition comprising from about 2 mg to about 800 mg of paraxanthine.
45. The method of clause 44, wherein the stress related disorder is selected from depression, major depressive disorder (MDD), anxiety disorder, panic disorder (episodic paroxysmal anxiety), panic attack, obsessive compulsive disorder, social anxiety disorder, phobic anxiety disorders, posttraumatic stress disorder (PTSD), acute stress disorder, obsessive compulsive disorder, hypertension, and inflammatory bowel syndrome.
46. A method of attenuating fatigue associated oxidative stress in a subject in need thereof comprising:
administering to the subject a composition comprising from about 2 mg to about 800 mg of paraxanthine.
47. The method of clause 46, wherein paraxanthine is present in the composition in amount from about 20 mg to about 600 mg.
48. The method of clause 47, wherein paraxanthine is present in the composition in amount from about 50 mg to about 400 mg.
49. The method of clause 46, wherein administration of the composition to the subject increases levels of catalase in the subject.
50. The method of clause 49, wherein catalase levels are increased in the subject by from about 5% to about 70%.
51. The method clause 46, wherein administration of the composition to the subject increases glutathione in the subject.
52. The method of clause 51, wherein glutathione levels are increased in the subject by from about 3% to about 30%.
53. A method of attenuating the effects of chronic stress on a subject comprising:
administering to the subject a composition comprising from about 2 mg to about 800 mg of paraxanthine.
54. The method of clause 53, wherein paraxanthine is present in the composition in amount from about 20 mg to about 600 mg.
55. The method of clause 53, wherein paraxanthine is present in the composition in amount from about 50 mg to about 400 mg.
56. The method of clause 53, wherein the composition further comprises an anti-cortisol agent.
57. The method of clause 56, wherein the anti-cortisol agent is one or more of: phosphatidylserine, rhodiola, ashwagandha, magnolia, holy basil, Omega 3s, and/or l-theanine.
58. The method of any of clauses 53-57, wherein administration of the composition to the subject increases BDNF level in the subject.
59. The method of clause 58, wherein BDNF levels are increased by from about 5% to about 40%.
60. The method of clause 59, wherein BDNF levels are increased by at least about 15%.
61. The method of any of clauses 53-60, wherein administration of the composition to the subject increases levels of catalase and/or glutathione in the subject.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

### Mental Fatigue Induced by Physical Stress

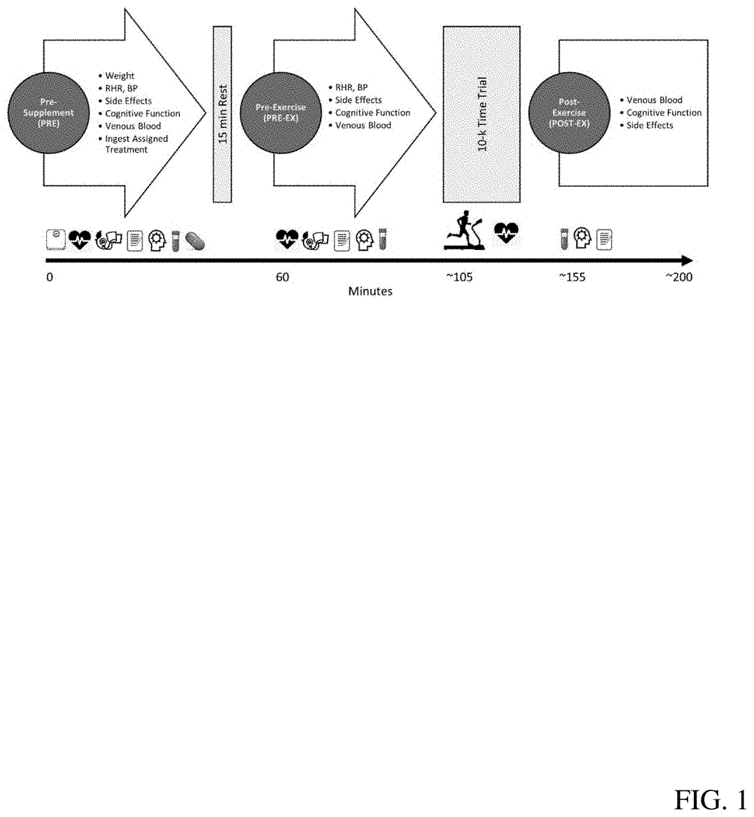

Trained runners from local running and triathlon clubs and races were recruited to participate in this study. Cognitive performance was assessed prior and after a mentally fatiguing endurance run. Eligibility criteria included healthy trained runners or triathletes between 18-40 years of age, current (>6 months) history of run training, and documented evidence that they averaged 8 minute/mile or less running pace during a recent competition (e.g., completing a 5-km road race or marathon). Qualified runners were invited to attend a familiarization session which provided an overview of the study and participants informed consent to participate in the study. A total of 32 individuals responded to study advertisements and were assessed for eligibility, 28 completed a familiarization session, 13 met eligibility requirements, agreed to participant in the study and were randomized into treatments. 1 subject dropped out of the study due to not complying with the study protocol. 12 subjects (11 males, 1 female) ultimately finished the trial. Volunteers visited the lab a total of five times including one familiarization session and four experimental sessions. During the familiarization session, participants were explained the study protocol, provided informed consent, answered a medical questionnaire, and had height, weight, resting heart rate, and resting blood pressure. Males then had body composition determined using dual energy X-ray absorptiometry (DXA) while females took a urine pregnancy test prior to the DXA scan to verify they were not pregnant. Participants then took each cognitive function test three times to familiarize them to the tests and establish test reliability. Volunteers practiced running on the treadmill to be used in the study at race pace. Once completed, the participants then performed a graded maximal cardiopulmonary (VO2 peak) treadmill test to determine peak heart rate and aerobic capacity. Participants were also asked to maintain usual eating habits and avoid new dietary supplements for the duration of the study. Participants were also asked to prepare for each testing session as they would a 10-km road race and refrain from vigorous physical activity, alcohol intake, and over-the-counter medications for 24 hours as well as fast for 8-12 hours prior to reporting to the lab. FIG. 1 shows the timeline of tests performed during each experimental testing session. Upon arriving at the lab, participants had weight, resting heart rate, and blood pressure determined. Participants then completed a side effects questionnaire, performed two cognitive function tests, the Psychomotor Vigilance Task Test (PVTT), a test that objectively assesses fatigue-related changes in alertness, and the Berg-Wisconsin Card Sorting Test (BCST), donated a fasting blood sample, and then ingested 1 of 4 randomly assigned oral supplements (PRE). Supplements were administered in a double blind, randomized, and crossover manner using a Balanced Latin Square method to counterbalance the order of treatment administration. Treatments included (1) 400 mg of placebo (PL, wheat flour, Shandong Bailong Chuangyuan Bio-tec Co. Ltd., Dezhou, China); (2) 200 mg of PL+200 mg of Caffeine (CSPC Innovation Pharmaceutical Co. Ltd., Shi Jiazhuang, China); (3) 200 mg of PL+200 mg of PX (ENFINITY™, Ingenious Ingredients, L.P. Lewisville, Tex., USA) or (4) 200 mg CA+200 mg of PX (CA+PX). Supplements were prepared using good manufacturing practices and certified for content. Supplements were the same size and appearance and packaged in generically labeled bottles for administration in a double-blind manner. Participants ingested one capsule with 8-ounces of water of the assigned treatment. Participants then rested for 15-minutes and repeated these tests. Volunteers then performed a 10-km run at their self-determined pace. Heart rate was obtained every kilometer during the run. Once completed, participants donated a venous blood sample. Participants then completed the cognitive function tests and the side effects questionnaire. After each testing session, participants observed a 7 to 14-day washout period and then repeated the protocol in identical fashion while ingesting remaining treatments in a randomized manner.

### Results:

### BCST (Berg-Wisconsin Card Sorting Test)

Paraxanthine supplementation increased the number of correct responses in the BCST by 5.7% compared to pre-exhaustive exercise, and reduced the number of errors by 1.5%, reduced the number or preservative errors by 4.6%, and the number of preservative errors (PAR rules) by 10.6%. In contrast, caffeine did not change the number of correct answers, however, increased the number of errors by 19.4%, increased the number or preservative errors by 15.9%, and the number of preservative errors (PAR rules) by 23.3%. Combining paraxanthine with caffeine improved correct responses and reduced errors in comparison to caffeine alone.

### PVTT (Psychomotor Vigilance Task Test)

Paraxanthine improved reaction time in the PVT Test by 7.3% during early trials (trial 2), by 10.9% in middle trials (trial 10), and by 10.9% in late trials (trial 20) compared to pre-exhaustive exercise. In contrast, the placebo group showed mental fatigue by worsening of reaction times by 31.7% in trial 10, and 8.3% in trial 20. Combining paraxanthine with caffeine improved reaction time in middle and late trials compared to placebo. Compared to caffeine, paraxanthine showed greater improvements in reaction time in trial 2 by 25%, in trial 10 by 44%, and in trial 20 by 12%.

### Example 2

### Mental Fatigue Induced by Mental Stress

Male, 51, consumed 200 mg of paraxanthine or 200 ng of caffeine or placebo, separated by one-week wash-out, prior to performing a serial calculation test, the Uchida-Kraepelin test (UKT), that is designed to measure mental fatigue. The test has been standardized and is widely used in clinical psychology, psychiatry, and occupational mental health practice. In this test, a subject is instructed to perform serial calculation of random numbers printed on a paper for 15 minutes. After a 5-minute rest, another 15 minutes of calculations are performed. The test subject is instructed to compute as many calculations as possible. Performance is assessed by 1-minute intervals.

Results: after consumption of placebo, performance was constant during the first 15 minutes. After the 5-minute rest, the initial performance increased over the mean performance of the first 15-minutes, but gradually and linearly decreases in the latter 15 minutes showing mental fatigue. After consumption of paraxanthine, mean performance during the first 15-minutes increased by 15%, mean performance during the second 15-minutes increased 21%, and paraxanthine prevented mental fatigue, as measured by the gradual decrease in performance during the second 15-minutes in the placebo group. In contrast, caffeine increased performance during the first 15-minutes by a significantly lower amount, 7%, and failed to prevent metal fatigue during the second 15-minutes of serial calculations.

### Example 3

### Paraxanthine Increases BDNF Levels in Young and Old Rats

Brain-derived neurotrophic factor (BDNF) is a protein found in the brain and spinal cord that promotes the survival of nerve cells by playing a role in the growth, maturation, and maintenance of these cells. Paraxanthine supplementation significantly increased BDNF levels in young rats from 775.04±29.59 pg/mL in the control group to 828.05±35.03 pg/mL in the low-dose paraxanthine group, and to 939.15±42.34 pg/mL in the high-dose paraxanthine group. Aging reduced BDNF levels (from 775.04±29.59 pg/mL to 732.16±16.51 pg/mL) and supplementation of old rats with paraxanthine significantly increased BDNF levels to 776.40±18.54 pg/mL in the low-dose paraxanthine group, and to 863.40±35.21 pg/mL in the high-dose paraxanthine group.

### Paraxanthine Increases Catalase and Glutathione Levels in Young and Old Rats

Physical and psychological stress are associated with increased oxidant production and oxidative damage. Catalase is a heme enzyme that is present in the peroxisome of nearly all aerobic cells. Catalase converts the reactive oxygen species hydrogen peroxide to water and oxygen and thereby mitigates the toxic effects of hydrogen peroxide. Glutathione is an antioxidant.

Paraxanthine supplementation significantly increased catalase levels in young rats from 27.76±1.21 U/mL in the control group to 30.24±1.58 U/mL in the low-dose paraxanthine group, and to 38.91±2.65 U/mL in the high-dose paraxanthine group. Aging reduced catalase levels (from 21.85±1.35 μg/mL to 26.15±1.03 U/mL) and supplementation of old rats with paraxanthine significantly increased catalase levels to 29.34±1.50 U/mL in the low-dose paraxanthine group, and to 36.84±1.68 U/mL in the high-dose paraxanthine group.

Paraxanthine supplementation significantly increased glutathione levels in young rats from 21.85±1.35 μg/mL in the control group to 24.04±1.74 μg/mL in the low-dose paraxanthine group, and to 34.41±1.97 μg/mL in the high-dose paraxanthine group. Aging reduced glutathione levels (from 21.85±1.35 μg/mL to 19.74±1.14 μg/mL) and supplementation of old rats with paraxanthine significantly increased glutathione levels to 22.27±1.00 μg/mL in the low-dose paraxanthine group, and to 27.49±0.84 μg/mL in the high-dose paraxanthine group.

What is claimed is:

1. A method for attenuating stress-induced mental fatigue in subject in need thereof, comprising:
administering to the subject a composition comprising from about 2 mg to about 800 mg of paraxanthine.

2. The method of claim 1, wherein paraxanthine is present in the composition in amount from about 50 mg to about 400 mg.

3. The method of claim 1, wherein the stress is physical stress.

4. The method of claim 1, wherein the stress is psychological stress.

5. The method of claim 4, wherein the stress is an acute stress.

6. The method of claim 4, wherein the stress is a chronic stress.

7. The method of claim 1, wherein the composition further comprises one or more additional active agents and wherein in the one or more active agents are selected from: L-Theanine, a blood flow enhancing agent, a nitric oxide stimulating agent, L-Arginine, L-Citrulline, Ginkgo Biloba, choline, phosphatidylserine, creatine, omega-3 fatty acids, beta-alanine; L-tyrosine; N-Acetyl-L-tyrosine; L-Ornithine; L-ornithine-L-aspartate; Melissa officinalis (Lemon Balm); pyrroloquinoline quinone; L-taurine; arginine alpha-ketoglutarate, and blueberries.

8. The method of claim 1, wherein the composition further comprises an anti-cortisol agent and one or more anti-cortisol agent is selected from: phosphatidylserine, rhodiola, ashwagandha, magnolia, holy basil, omega 3s, and/or l-theanine.

9. The method of claim 1, wherein the composition is administered prior to the onset of stress in the subject.

10. The method of claim 1, wherein the composition does not include caffeine and wherein the subject abstains from consuming caffeine during the steps of the method.

11. A nutritional supplement to prevent mental fatigue induced by a stressor comprising from about 2 mg to about 800 mg paraxanthine and a nutraceutically acceptable carrier thereof.

12. The nutritional supplement of claim 11 wherein the paraxanthine is present in amount from about 50 mg to about 400 mg.

13. The nutritional supplement of claim 12, further comprising one or more additional active agents and wherein in the one or more active agents are selected from: L-Theanine, a blood flow enhancing agent, a nitric oxide stimulating agent, L-Arginine, L-Citrulline, Ginkgo Biloba, choline, phosphatidylserine, creatine, omega-3 fatty acids, beta-alanine; L-tyrosine; N-Acetyl-L-tyrosine; L-Ornithine; L-ornithine-L-aspartate; Melissa officinalis (Lemon Balm); pyrroloquinoline quinone; L-taurine; arginine alpha-ketoglutarate, and blueberries.

14. The nutritional supplement of claim 11, further comprising an anti-cortisol agent and wherein the anti-cortisol agent is one or more of: phosphatidylserine, rhodiola, ashwagandha, magnolia, holy basil, omega 3s, and/or l-theanine.

15. A method of enhancing the stress resiliency in a subject in need thereof, comprising: administering to the subject a composition comprising from about 2 mg to about 800 mg of paraxanthine.

16. The method of claim 15, wherein administration of the composition to the subject increases resiliency exercise-induced stress.

17. The method of claim 16, wherein administration of the composition to the subject increases resiliency psychologically-induced stress.

18. The method of claim 15, wherein administration of the composition to the subject increases BDNF level in the subject and wherein BDNF levels are increased by from about 5% to about 40%.

19. The method of claim 15, wherein administration of the composition to the subject increases levels of catalase and/or glutathione in the subject.

20. The method of a claim 15, wherein increasing resiliency to stress decreases susceptibility of the subject to a stress related disorder selected from: depression, major depressive disorder (MDD), anxiety disorder, panic disorder (episodic paroxysmal anxiety), panic attack, obsessive compulsive disorder, social anxiety disorder, phobic anxiety disorders, posttraumatic stress disorder (PTSD), acute stress disorder, obsessive compulsive disorder, hypertension, and inflammatory bowel syndrome.
